(19) United States
(12) Patent Application Publication
(10) Pub. No.: US 2011/0112548 A1
(43) Pub. Date: May 12, 2011

(54) METHODS AND SYSTEMS FOR REMOVAL OF IMPLANTABLE INTRAVASCULAR DEVICES

(76) Inventors: Daniel Fifer, Windsor, CA (US); Richard Glenn, Santa Rosa, CA (US); Jeffrey Smith, Petaluma, CA (US); Geoff Orth, Sebastopol, CA (US); Michael Williams, Santa Rosa, CA (US); William Sanders, Chapel Hill, NC (US); Earl Bardsley, Raleigh, NC (US); Wendy Graczyk, Raleigh, NC (US)

(21) Appl. No.: 12/941,796

(22) Filed: Nov. 8, 2010

Related U.S. Application Data
(60) Provisional application No. 61/258,773, filed on Nov. 6, 2009.

(51) Int. Cl. A61B 19/00

(52) U.S. Cl. 606/129

(57) ABSTRACT

Improved methods, systems and apparatuses for extracting devices implanted within the vasculature of a patient are provided, including a method of extracting an intravascular device anchored in a vasculature of a patient, the device including a device body and a lead coupled to the device body, the lead having a distal end including an electrode, the distal end being fixed in a heart of the patient, the method comprising forming an incision in the vasculature, disconnecting the lead from the device body, removing the distal end of the lead from the heart, withdrawing the lead from the patient, disconnecting the device body from an anchor, and withdrawing the device body from the patient via the incision.

### RELATED APPLICATION

The present invention claims priority to U.S. Provisional Patent Application No. 61/258,773, entitled “Methods and Systems for Extraction of Implantable Intravascular Devices,” filed Nov. 6, 2009, which is hereby incorporated by reference.

### FIELD OF THE INVENTION

The present invention relates generally to implantable intravascular devices, and more particularly to solutions for extraction or removal of such intravascular devices.

### BACKGROUND OF THE INVENTION

Implantable devices that provide long-term active therapies such as pacemakers, defibrillators, and implantable cardioverter defibrillators (“ICDs”) have been successfully implanted in patients for years for treatment of heart rhythm conditions. Pacemakers are implanted to detect periods of bradycardia and deliver low energy electrical stimuli to increase the heart rate. ICDs are implanted in patients to cardiovert or defibrillate the heart by delivering high-energy electrical stimuli to slow or reset the heart rate in the event a ventricular tachycardia (VT) or ventricular fibrillation (VF) is detected. Another type of implantable device detects an atrial fibrillation (AF) episode and delivers an electrical stimuli to the atria to restore electrical coordination between the upper and lower chambers of the heart. The current generation for all of these implantable cardiac rhythm management (CRM) devices are typically can-shaped devices implanted under the skin that deliver electrical stimuli via leads that are implanted in the heart via the patient's vascular system.

These conventional CRM devices are implanted subcutaneously, typically in the pectoral region, and include one or more conventional leads in the form of elongated, floppy lead bodies that insulate, seal and protect one or more conductors which transmit electrical signals for stimulating and/or sensing between the pulse generator and one or more electrodes on the lead. The one or more leads associated with a conventional CRM device are typically not integrated with the device; instead, a header is provided on the device for connecting the one or more leads to the device. Such a connection arrangement between the conventional device and the lead allows for access to a lumen within the lead from outside the body of the patient via the proximal end of the lead that is superior to the heart to aid in implantation and removal.

Implantation of the one or more intravascular leads for a conventional CRM device involves delivery of the lead to a desired location, followed by fixation of the lead. For a CRM device implanted subcutaneously in the pectoral region, the most common path for delivering the lead into the heart begins at a transvenous incision into the subclavian vein, through the superior vena cava, and down into a chamber of the heart. The lead tip on the distal end of the lead is affixed in, on, or near the heart, depending on the desired treatment. Most intravascular cardiac leads for conventional CRM devices are guided through the vasculature with use of a stylet that is inserted into the lumen within the lead body accessed via the proximal end of the lead that is located outside the body of the patient until the final steps of the procedure, with the stylet used to direct the distal end of the lead into the desired position. Implantation of the device typically follows implantation of the lead. The proximal end of the lead is connected to the device at the header, and the device is then secured in the patient.

It may be necessary to explant a CRM device and the associated lead(s) from the patient, for example due to infection, or fractures in the lead conductor or insulation. To do so, the device is removed from the pectoral region of the patient, and the lead is disconnected from the header of the device. Once disconnected from the CRM device, the proximal end of the lead presents a free end that can be conveniently accessed and utilized to extract the lead. In one approach, a cutting tool is introduced into the central lumen of the lead via the disconnected free end of the lead. In another approach, a cutting tool can be advanced over the free end of the lead body and advanced over or along the lead to a position proximate the lead tip. When positioned proximate the lead tip, the cutting device is used to sever the lead body from the tip, and the lead body can be extracted, leaving the tip implanted in the heart. Alternatively, the cutting tool can be used to cut away scar tissue from the area surrounding the tip. In a further approach, a catheter is introduced over the free end of the lead body and advanced toward the lead tip. The catheter is used to provide traction for pulling the lead from the heart. Laser removal techniques involve the use of a laser catheter advanced over the lead body to a position proximate the lead tip, wherein laser energy is applied to remove any scar tissue surrounding the tip. The lead is then free and can be removed through the sheath.

Next generation long-term active implantable devices may take the form of elongated intravascular devices that are implanted entirely within the patient's vascular system, instead of under the skin. Examples of these implantable intravascular devices (IIDs) are described, for example, in U.S. Pat. Nos. 7,082,336, 7,529,589, 7,617,007, and 7,363,082, all assigned to Synecor, LLC. These devices contain electric circuitry and/or electronic components that must be hermetically sealed to prevent damage to the electronic components and the release of contaminants into the bloodstream. Due to the length of these implantable devices, which in some cases can be approximately 10-60 cm in length, the devices must be flexible enough to move through the vasculature while being sufficiently rigid to protect the internal components.

In some embodiments, these implantable intravascular devices include cardiac leads that are coupled to one end of the elongated device body. The lead can be looped from the inferior end of the elongated device body residing in the vena cava, for example, up to the entrance into the right atrium and into the right ventricle. The lead can also be fixated in one or more other chambers of the heart, namely the right atrium, left atrium, left ventricle, or within a vessel of the heart, e.g. coronary sinus or middle cardiac vein. In these embodiments, the cardiac lead of an IID is unlike a cardiac lead for a conventional CRM device in that the proximal end of the lead is generally unavailable for access to aid in the implantation or explantation of the lead.

Because of the numerous differences between conventional CRM devices and IIDs, removal tools and techniques for conventional CRM devices and associated leads are not necessarily applicable to implantable intravascular devices. For example, as IIDs are anchored within the vasculature of the patient, conventional CRM device removal techniques and tools are not necessarily applicable.

Additionally, lead removal techniques for conventional implantable CRM devices typically rely on free access to the proximal end of the lead body, which is achieved by creating a small incision in the pectoral region of the patient, removing the CRM device, and simply unplugging the lead from the header of the device outside of the body of the patient. Such access to the lead body may be difficult or impossible with implantable intravascular devices, as the lead is wholly within the vasculature and heart of the patient, and in some arrangements the lead may be integrated with the body of the IID.

An approach to facilitating removal of an IID is presented in U.S. Published Application No. 2008/0147168, which is assigned to the assignee of the present invention. Embodiments described therein feature an implantable intravascular device having a detachable tether arrangement positioned proximate an end of the elongated body portion of the IID. An anchoring arrangement is provided to secure the tether portion within the vasculature. Separation of the IID from the tether permits removal of the IID body while leaving the tether portion and anchoring arrangement within the vessel.

One proposed solution for the introduction, fixation and removal of an IID lead is presented in U.S. Published Application No. 2009/0198251, which is assigned to the assignee of the present invention. In one embodiment, a system for implanting a lead of an implantable intravascular device is provided, wherein the lead includes a proximal end attached to the IID and an electrode portion proximate a distal end. The system includes a steerable guide catheter having a torqueable driver therein and a catheter tip, and a sidecar apparatus having a first bore configured to receive the lead and a second bore including a bulkhead adapted to couple to the guide catheter tip, the second bore being substantially parallel to and axially offset from the first bore. The second bore of the sidecar apparatus includes a selectively deployable fixation arrangement.

To extract the lead, a sheath or other tool may be used to provide counter-traction for grasping the lead. The lead body may first be severed near its connection to the implantable intravascular device, and a sheath may then be advanced over the lead body until the sheath abuts the sidecar. The sheath is used for counter-traction while the lead body is grasped with a tool and pulled from the sidecar, disconnecting the lead from the sidecar apparatus. Alternatively, a tool may be advanced alongside the lead body, whether the lead is severed from the IID or not, and positioned against the sidecar. The tool is then used for counter-traction while the lead body is grasped with a tool and pulled from the sidecar, disconnecting the lead from the sidecar apparatus.

An alternative intravascular medical device (IVMD) is described in U.S. Pat. Nos. 7,519,424, 7,616,992 and 7,627,376 and U.S. Published Application No. 2009/0198295, all of which are assigned to Medtronic, Inc. Embodiments of IVMD's described therein generally include a housing, one or more leads extending from the distal end of the housing, and a tether extending from the proximal end of the housing. The IVMD is implanted through a transvascular incision, such as in the subclavian vein. The lead is guided (with a steerable stylet having a grasper mechanism or inserted into a lumen that runs throughout the leads and housing, or using a catheter/guidewire arrangement) into the subclavian vein and toward the heart where it is fixated. In this respect, the lead for the IVMD is implanted in a similar fashion as a lead for a conventional CRM device. The housing and tether of the IVMD follow the lead into the vasculature during implantation. The tether preferably includes a T-shaped anchor member configured to remain outside of the vasculature, with the tether and optional anchor member being sutured or otherwise secured to tissue outside the vasculature and proximate the incision site. Removal of the IVMD is contemplated through the same incision used for implantation. For instance, the extravascular anchor is located, access is created in the vessel, and the tether portion, housing, and lead are all removed through this access by use of the proximal end of the IVMD being exposed outside of the body of the patient.

While the above approaches for removal of implantable intravascular devices and associated leads are improvements or adaptations of methods and devices employed with conventional CRM devices, a need still exists for further improved extraction methods and tools for IIDs and associated leads, especially for IIDs that extend into the inferior vena cava.

### SUMMARY OF THE INVENTION

In one embodiment, a method of extracting an intravascular device anchored in a vasculature of a patient is provided, the device including a device body and a lead coupled to the device body, the lead having a distal end including an electrode, the distal end being fixed in a heart of the patient, the method comprising forming an incision in the vasculature, disconnecting the lead from the device body, removing the distal end of the lead from the heart, withdrawing the lead from the patient, disconnecting the device body from an anchor, and withdrawing the device body from the patient via the incision.

In another embodiment, a system for extracting an intravascular device anchored in a vasculature of a patient is provided, the device including a device body and a lead coupled to the device body, the lead having a distal end including an electrode, the distal end being fixed in a heart of the patient, the system comprising a safety sheath configured to be temporarily introduced into the vasculature of the patient, a lead snare having a closeable snare portion, the lead snare configured to releasably capture a portion of the lead, a lead cutter having a cutting mechanism, the lead cutter configured to sever the lead, a device snare having a closeable snare portion, the device snare configured to releasably grasp the device body of the intravascular device, and a tip cutter having a closeable loop portion, the loop portion configured to be advanceable over the device body.

In another embodiment, a method of extracting an intravascular device anchored in a vasculature of a patient is provided, the device including a device body and a lead coupled to the device body, the lead having a distal end including an electrode, the distal end being fixed in a heart of the patient, the method comprising providing a safety sheath, a snare, a lead cutter and a tip cutter, and providing instructions for extracting the intravascular device, including forming an incision in the vasculature, positioning the safety sheath within the vasculature and proximate the lead, grasping the lead with the snare and pulling a portion of the lead within the safety sheath, cutting the lead with the lead cutter, grasping the device body with the snare, advancing the tip cutter over the device body to a desired location, and cutting the device body to disconnect the device body from an anchor.

The above summary of the various embodiments of the invention is not intended to describe each illustrated embodiment or every implementation of the invention. This summary represents a simplified overview of certain aspects of the invention to facilitate a basic understanding of the invention and is not intended to identify key or critical elements of the invention or delineate the scope of the invention.

### BRIEF DESCRIPTION OF THE DRAWINGS

The invention may be more completely understood in consideration of the following detailed description of various embodiments of the invention in connection with the accompanying drawings, in which:

FIG. 1 depicts an exemplary IID.

FIGS. 2A-2C depict a safety sheath according to an embodiment of the invention.

FIGS. 3A-3B depict a lead snare according to an embodiment of the invention.

FIGS. 3C-3D depict a lead snare handle according to an embodiment of the invention.

FIG. 4A depicts a lead cutter according to an embodiment of the invention.

FIG. 4B depicts a lead cutter handle according to an embodiment of the invention.

FIGS. 5A-5C depict a lead cutter tip according to an embodiment of the invention.

FIGS. 6A-6E depict a device snare according to an embodiment of the invention.

FIGS. 7A-7B depict a tip cutter according to an embodiment of the invention.

FIG. 8A depicts a tip cutter handle according to an embodiment of the invention.

FIG. 8B depicts a cut away view of the tip cutter handle of FIG. 8A.

FIG. 8C depicts a tip cutter handle according to an embodiment of the invention.

FIG. 9 depicts a tip cutter according to an embodiment of the invention.

FIG. 10 depicts a tip cutter with a scissors-style handle according to an embodiment of the invention.

FIG. 11 depicts a tip cutter handle and a tip cutter inserted into an insertion sheath.

FIG. 12 depicts a lead snare being placed over an IID lead according to an embodiment of the invention.

FIG. 13 depicts an IID lead drawn into a safety sheath by a lead snare according to an embodiment of the invention.

FIG. 14 depicts a lead cutter positioned to cut an IID lead according to an embodiment of the invention.

FIG. 15 depicts a device snare, a severed lead, and tip cutter according to an embodiment of the invention.

FIGS. 16A-16B depict a tip cutter disposed around an IID.

FIGS. 17A-17C depict a tip cutter disposed around an IID tip.

FIGS. 17D-17F depict a tip cutter cutting an IID tip according to an embodiment of the invention.

FIG. 18 depicts a flow chart of a method of removing an IID according to an embodiment of the invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

### DETAILED DESCRIPTION OF THE DRAWINGS

In the following detailed description of the present invention, numerous specific details are set forth in order to provide a thorough understanding of the present invention. However, one skilled in the art will recognize that the present invention may be practiced without these specific details. In other instances, well-known methods, procedures, and components have not been described in detail so as to not unnecessarily obscure aspects of the present invention.

The present invention generally comprises tools and methods for extracting an implantable intravascular device (IID) from a patient.

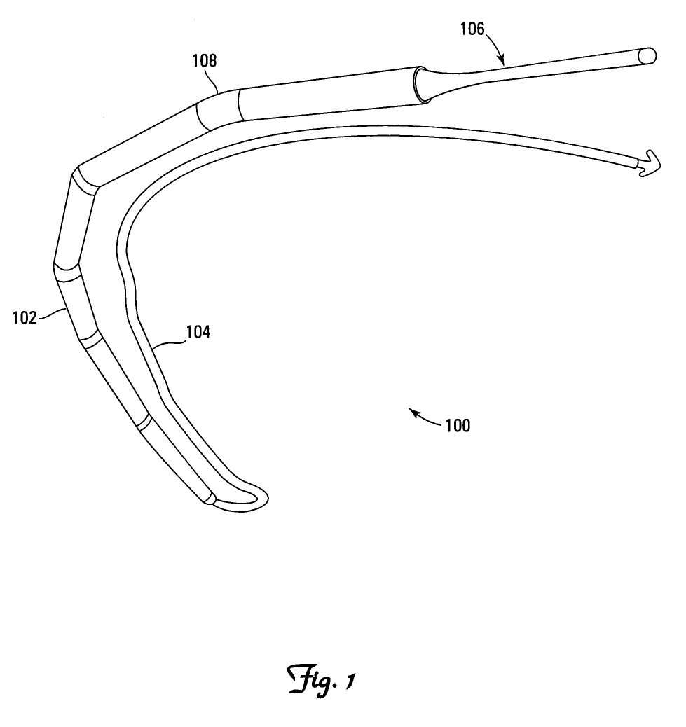

FIG. 1 depicts an exemplary IID 100 including a cylindrical housing 102, lead 104, tip 106, and articulations 108. The IID 100 generally includes components known in the art to be necessary to carry out the system functions. For example, the IID 100 may include a body with proximate and distal ends, housing one or more pulse generators, including associated batteries, capacitors, microprocessors, and circuitry for generating electrophysiological pulses for defibrillation, cardioversion and/or pacing. The IID 100 can also include detection circuitry for detecting arrhythmias or other abnormal activity of the heart. The specific components to be provided in the device will depend upon the application for the device, and specifically whether the device is intended to perform defibrillation, cardioversion and/or pacing along with its sensing functions.

The IID 100 comprises one or more elongated generally cylindrical housings 102, coupled by articulators 108, and proportioned to be passed into the vasculature and to be anchored within the patient's vasculature with minimal obstruction to blood flow. IID 100 also comprises at least one lead 104 configured to be attached to a desired treatment area. Suitable sites for the IID 100 can include, but are not limited to, the venous system using access through the right or left femoral vein or the subclavian or brachiocephalic veins, or the arterial system using access through one of the femoral arteries. Thus, the body of IID 100 preferably has a streamlined maximum cross sectional diameter which can be in the range of 3-15 mm or less, with a most preferred maximum cross-sectional diameter of 3-8 mm or less. The cross-sectional area of the device in the transverse direction (i.e. transecting the longitudinal axis) should be as small as possible while still accommodating the required components. The cross-section of the device (transecting the longitudinal axis) can have a circular cross-section, although other cross-sections including crescent, flattened, or elliptical cross-sections can also be used. It can be desirable to provide the device with a smooth continuous contour so as to avoid voids or recesses that could encourage thrombus formation on the device.

Additional information pertaining generally to the construction, arrangement and function of an IID suitable for use in accordance with the present invention can be found in U.S. Pending application Ser. Nos. 12/815,210 titled “Methods and Systems for Anti-Thrombotic Intravascular Implantable Devices”, filed Jun. 14, 2010, and 12/815,355 titled “Intravascular Implantable Device”, filed Jun. 14, 2010, U.S. Published Application Nos. 2007/0265673, 2008/0147168, 2008/0167702, 2009/0192579, and 2009/0198251, and U.S. Pat. Nos. 7,082,336, 7,363,082, 7,529,589, and 7,617,007, the disclosures of which are hereby incorporated by reference.

Tools for explanting an IID 100 generally include a safety sheath 120, lead snare 130, lead cutter 140, device snare 150, and tip cutter 160, which are each discussed in more detail below.

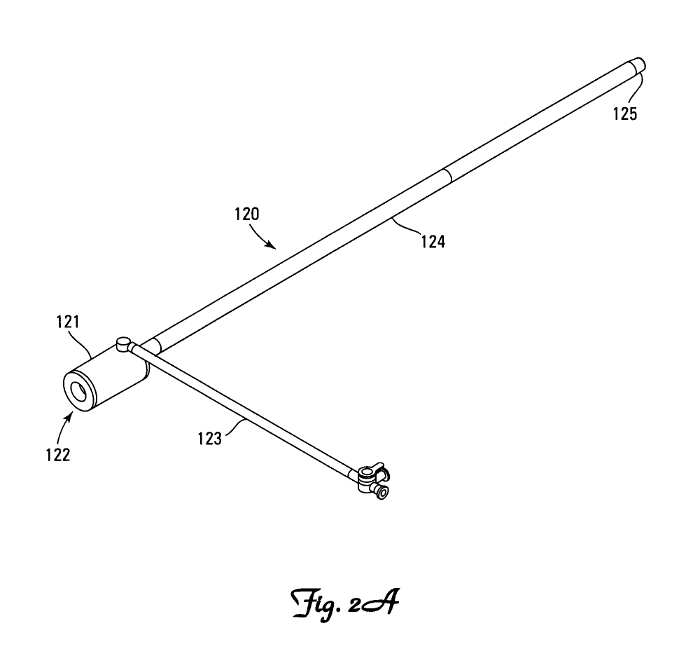

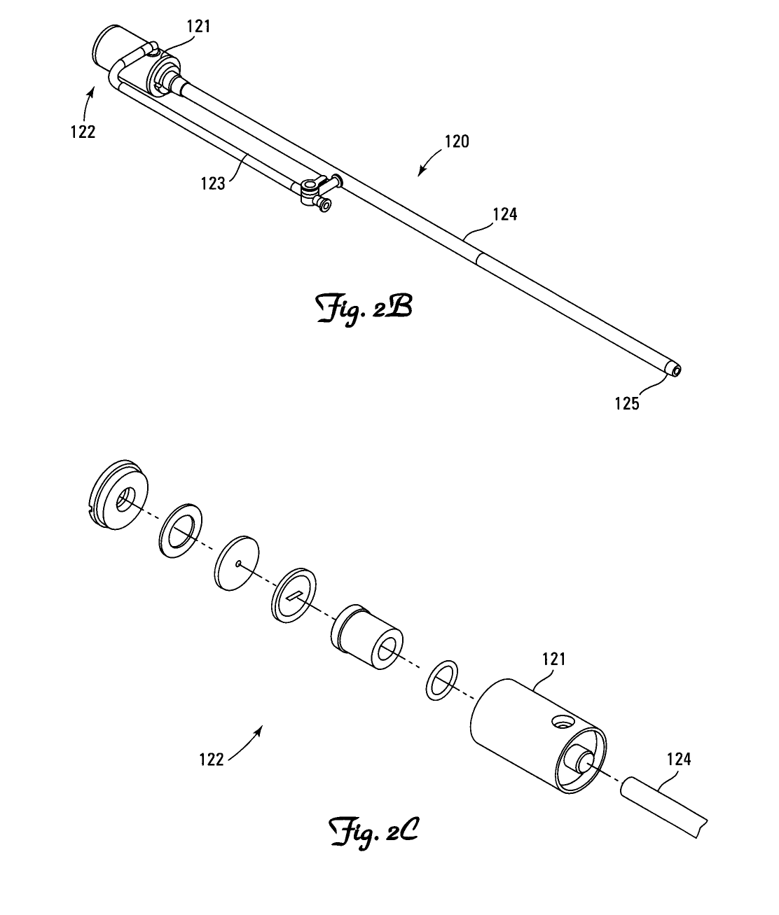

As depicted in FIGS. 2A and 2B, an embodiment of safety sheath 120 comprises a proximal handle portion 121, a sealing/valve means 122, flush port 123, tubular shaft 124, and tip portion 125. Handle portion 121 is configured to remain outside of the patient, and provide operating control of sealing means 122. Sealing means 122 can comprise an iris or box fold valve, or other arrangement configured to permit insertion of tools into the patient while preventing blood loss. One example of the various, generally annular, components of sealing means 122, disposed in the proximal handle portion 121, are depicted in FIG. 2C.

In one embodiment tubular shaft 124 can have a diameter of approximately twenty-two French, although it will be appreciated that smaller or larger diameters can be used without deviating from the scope of the invention. Shaft 124 can include a coating or be constructed of one or more lubricious materials so as to present a slippery surface for insertion and withdrawal of shaft 124 as well as insertion and withdrawal of the removal tools from within the shaft. At the distal end of shaft 124 is tip portion 125, which can include features to aid visualization during fluoroscopy. Handle portion can include secondary sealing means, including or similar to the hemostasis valve included in the introducer sheath discussed in U.S. Published Application No. 2009/0192579 which is incorporated by reference herein.

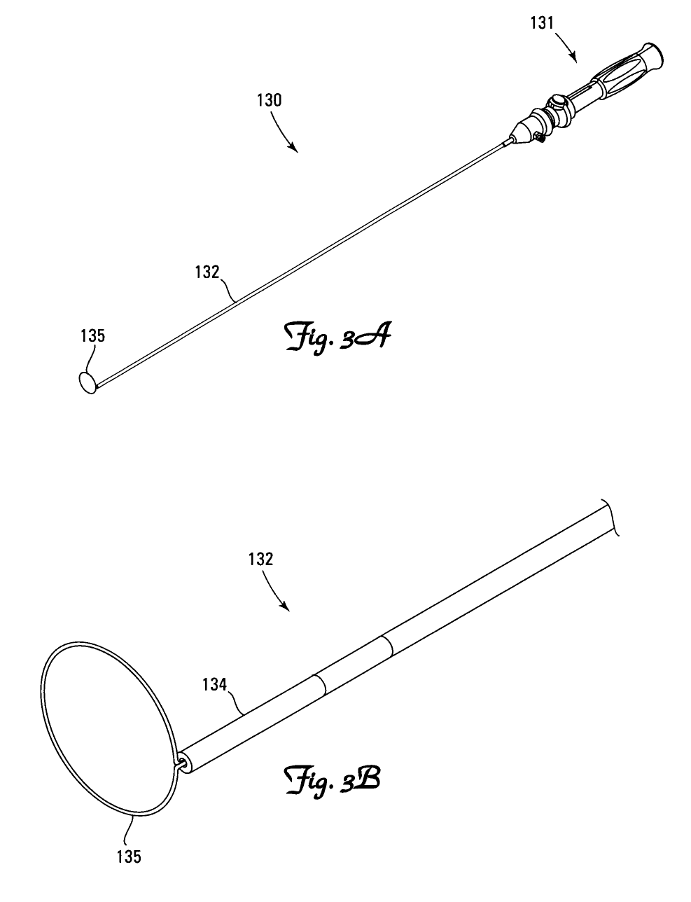

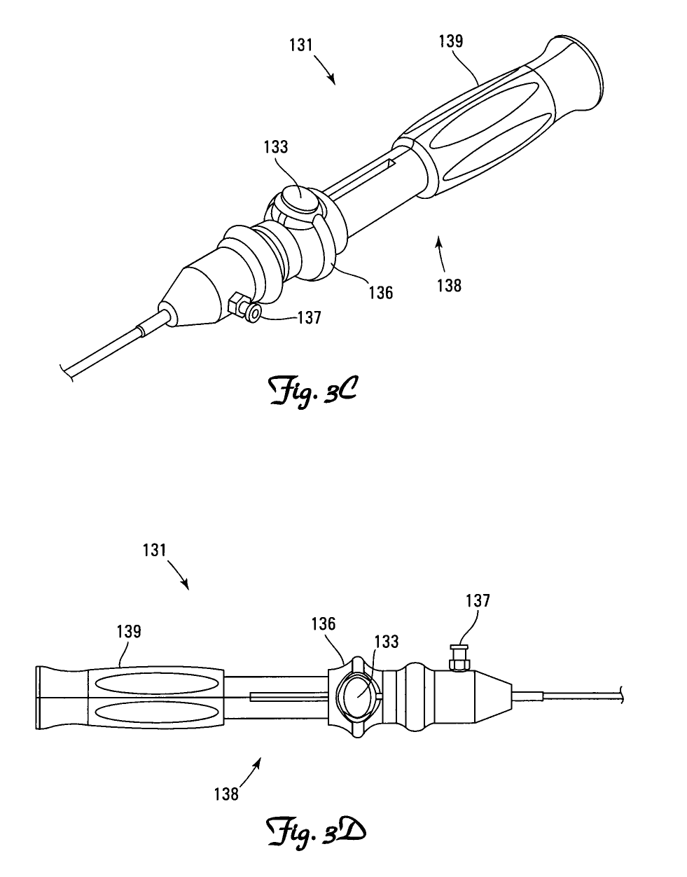

Referring to FIGS. 3A-3B, an embodiment of lead snare 130 comprises a deflectable snare, having a handle portion 131, catheter body 132, tip portion 134, loop portion 135. Handle portion 131 provides a mechanism for deflecting, steering or otherwise manipulating snare 130, an actuation slider 136 operable to open or close loop portion 135, as well as a locking mechanism.

In one embodiment, catheter body 132 can include a braided sheath extending the length of lead snare 130. Tip portion 134 includes a lumen for loop portion 135 to pass through and can provide a surface against which a lead can be grasped as the loop portion 135 is constricted. Loop portion 135 can be constructed from a shape memory alloy, such as Nitinol. As depicted in the FIG. 3B, loop portion 135 is pre-shaped such that when fully deployed (open) loop 135 is oriented transverse to catheter body 132. Loop portion 135 can include a protective coil (not visible in Figures) to prevent cutting into an object being grasped. Opening and closing of loop 135 is controlled by handle portion 131.

As depicted in FIGS. 3C and 3D, handle portion 131 of lead snare 130 can include an actuation slider 136 coupled to loop 135 that travels along a handle base 138. The handle base 138 can include a grip 139. By manipulating actuation slider 136 along a length of the handle portion 131 the snare can be actuated, opening or closing loop portion 135. Handle portion 131 can also include a flush port 137. An embodiment of handle portion 131 can include a locking mechanism (not depicted) to prevent an inadvertent actuation of the loop portion 135 and to lock actuation slider 136 at intervals along handle base 138. The locking mechanism can be released, allowing actuation slider 136 to travel along the length of handle base 138, when button 133 is depressed.

In one embodiment, lead snare 130 can also include an outer sleeve that can be longitudinally translatable along catheter body 132. Extending or retracting outer sleeve, while maintaining loop portion 135 in place, has the effect of closing or opening loop portion 135, and can be used as a quick check prior to actually closing loop portion 135. Lead snare 130 can also include protective coil disposed in or on the catheter body 132.

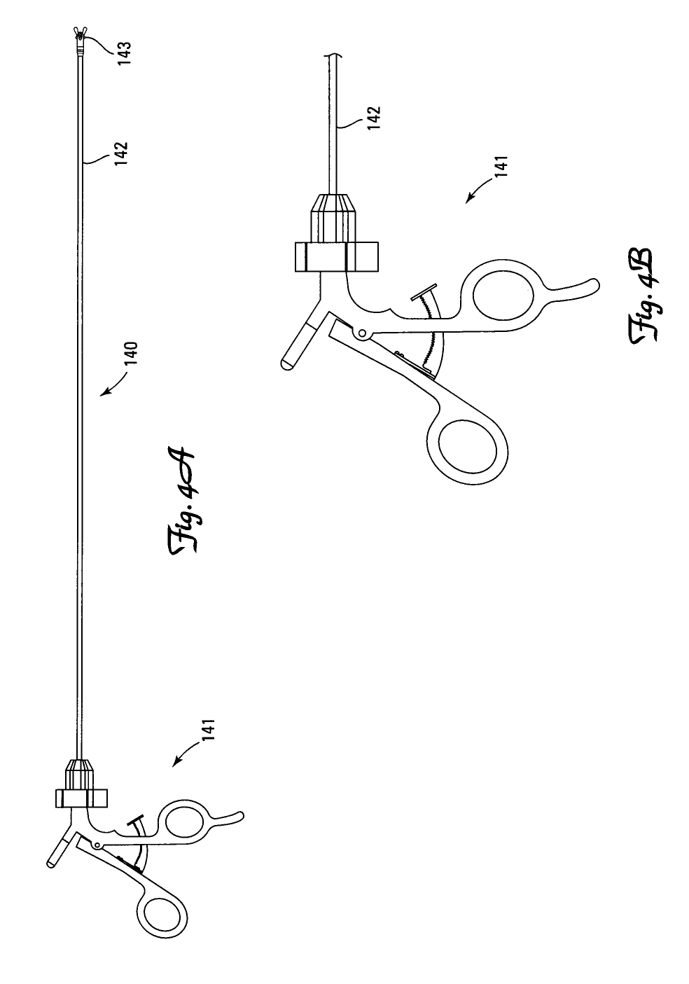

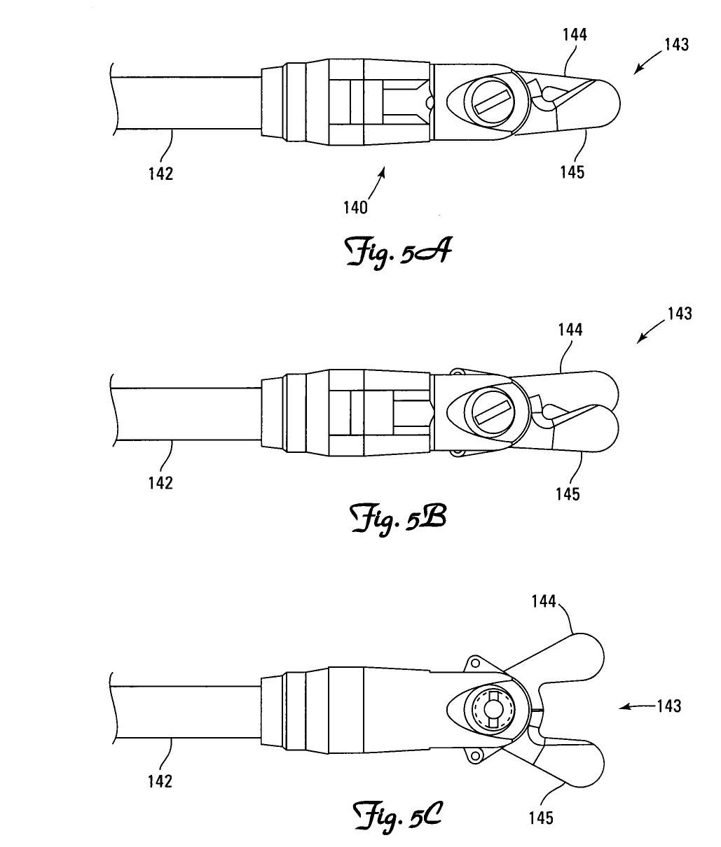

Referring to FIGS. 4A-4B and 5A-5C, an embodiment of lead cutter 140 includes a handle portion 141, catheter body 142, and tip portion 143 including blades 144, 145. Lead cutter 140 can comprise a bypass-style cutter (wherein blades 144, 145 at least partially overlap when making a cut) to insure a complete cut of a lead conductor is made, as well as provide visible confirmation under fluoroscopy.

Handle portion 141 can include a ratchet or locking mechanism to allow controlled actuation of blades 144, 145 and to prevent inadvertent actuation. A cable or wire can extend the length of the catheter body 142, coupling the handle portion 141 and the tip portion 143. The tip portion 143 can include a mechanism to translate the linear movement of the cable or wire into an opening or closing actuation movement of blades 144, 145. FIGS. 5A-5C depict blades 144, 145 in closed, partially open/closed, and open positions, respectively.

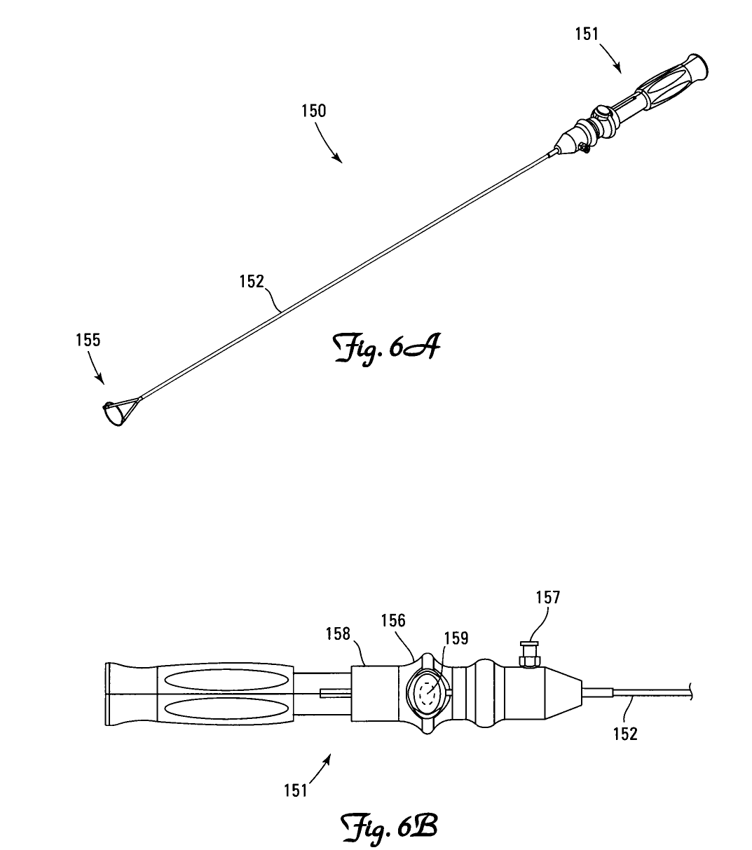

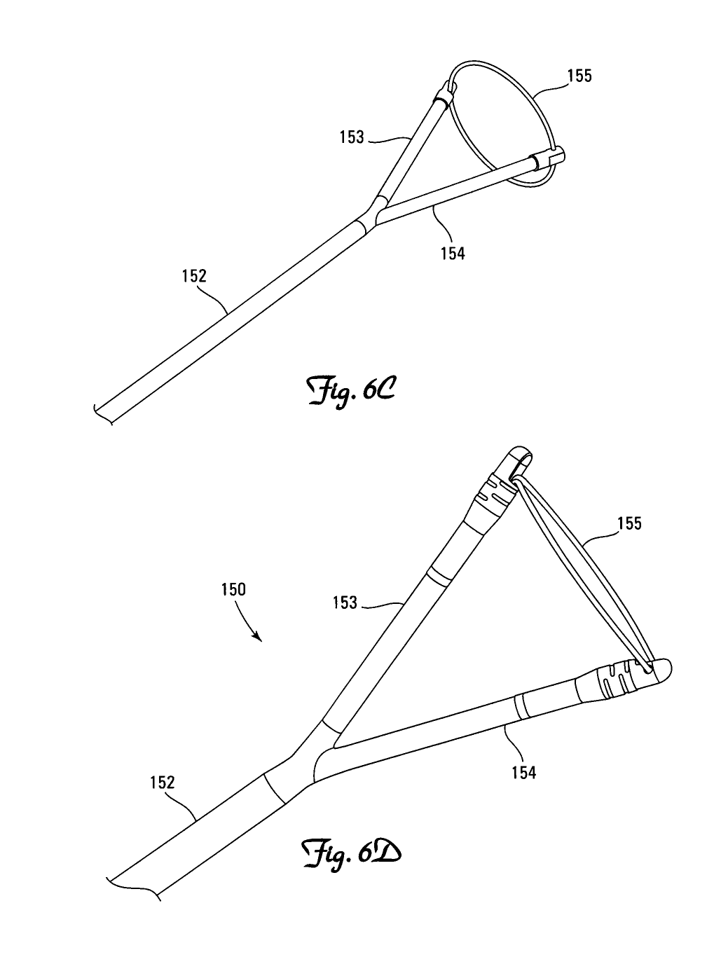

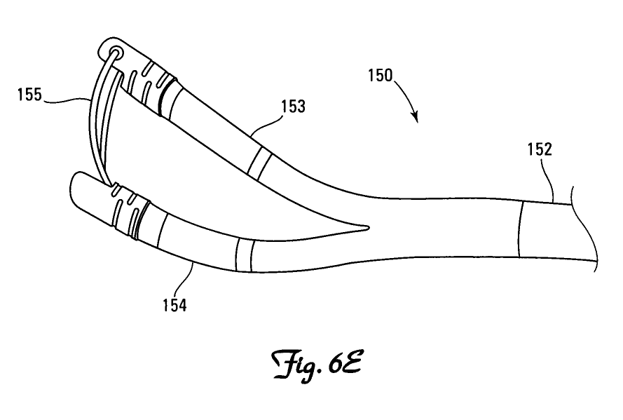

Referring to FIGS. 6A-6E, an embodiment of device snare 150 can comprise a deflectable shaft, having a handle portion 151, catheter body 152, arms 153, 154, and loop portion 155. Handle portion 151 can include means for deflecting, steering or otherwise manipulating snare 150, means for opening or closing loop portions 155, as well as a locking mechanism. Catheter body 152 can include an internal braid (not shown) extending the length of the device snare 150. Arms 153, 154 are somewhat flexible, such that when loop portion 155 is closed, arms 153 and 154 are proximate one another. When loop portion 155 is opened, arms 153 and 154 are angled away from each other, as depicted in FIGS. 6C-6E. Loop portion 155 is constructed from a shape memory alloy, such as Nitinol. Loop portion 155 can comprise a single wire, with each end passing into a single arm and the opposing arm featuring a passage for loop 155 to extend through. Alternatively, loop portion 155 can comprise a pair of wires each extending between arms 153 and 154. Each wire deploys into a semi-circular shape transverse to catheter body 152, such that the pair of deployed wires forms a generally circular snare as depicted in FIG. 6C. One of the pair of wires terminates at arm 153 and then passes into arm 154. The other of the pair of wires terminates at arm 154 and passes into 153.

As depicted in FIG. 6B, handle portion 151 can include an actuation slider 156 coupled to loop 155. By manipulating actuation slider 156 along a length of the handle portion 151 the snare can be actuated, opening or closing loop portion 155. Handle portion 151 can also include a flush port 157. In one embodiment handle portion 151 can include a locking mechanism to prevent an inadvertent actuation of the loop 155 and to secure slider 156 at various intervals along a length of the handle portion 151. The locking mechanism can be released, allowing slider 156 to travel along the length of handle portion 151 when button 159 is depressed. In one embodiment the handle portion 151 is generally similar to the handle portion 131 of lead snare 130 depicted in FIGS. 3C and 3D, the device snare 150 can include a spacer 158 to limit the range of motion of slider 156.

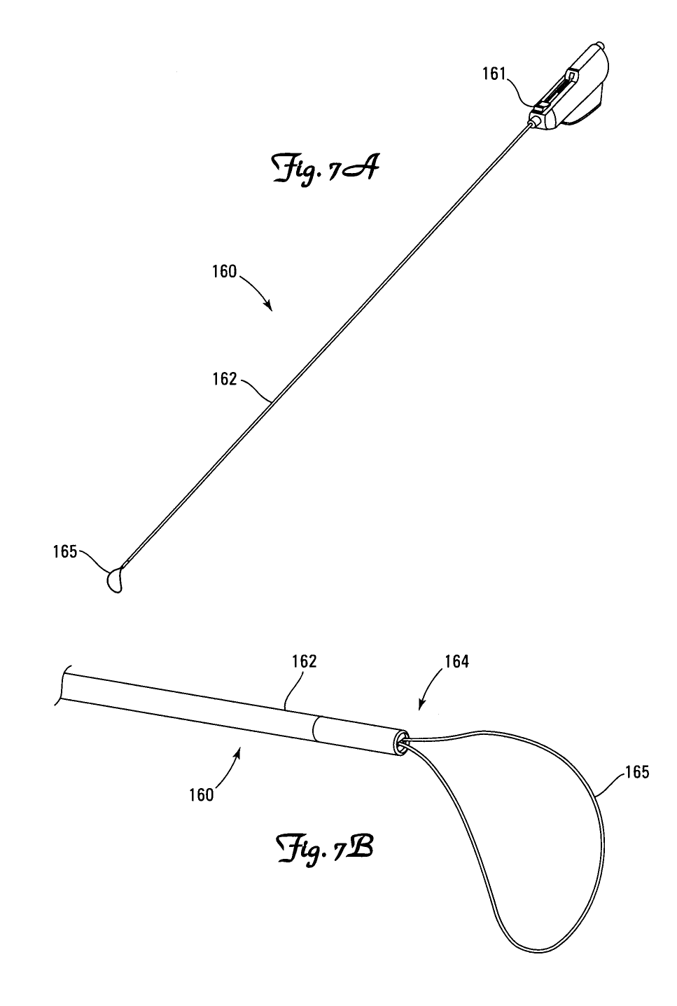

Referring to FIGS. 7A and 7B, an embodiment of tip cutter 160 includes a handle portion 161, catheter body 162, tip portion 164, and loop portion 165. Handle portion 161 includes means for manipulating cutter 160, means for opening or closing loop portion 165, as well as a safety cam locking mechanism. Catheter body 162 can include an internal support coil and/or braid (not shown) extending the length of tip cutter 160. Tip portion (or anvil) 164 includes a slot for loop portion 165 to pass through and presents a robust surface against which a tip or lead can be grasped and cut. Loop portion 165 is constructed from a shape memory alloy, such as Nitinol. As depicted in the Figures, loop portion 165 is pre-shaped such that when fully deployed (open) loop 165 is oriented transverse to catheter body 162. Opening and closing of loop 165 is controlled by handle portion 161.

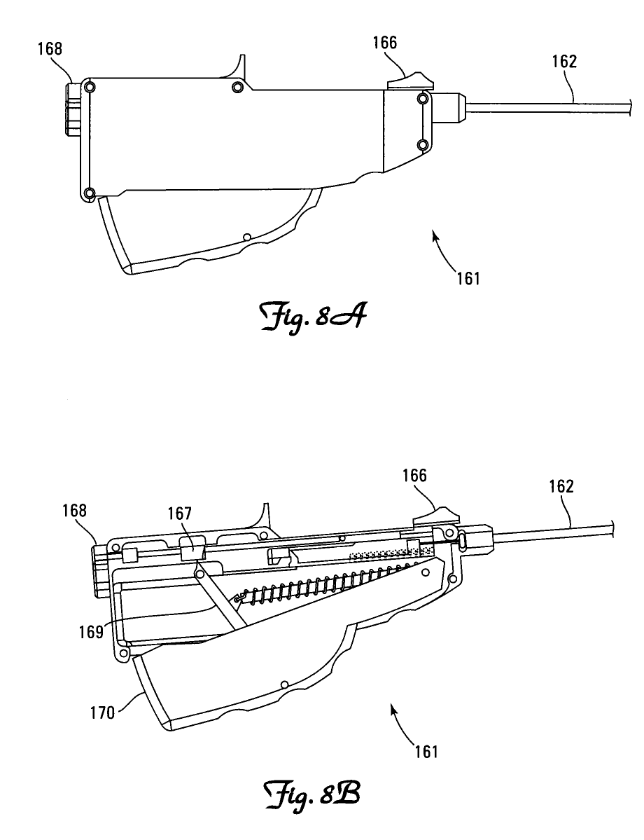

Referring to FIGS. 8A and 8B, an embodiment of handle portion 161 can include a slider 166 coupled to loop portion 165. The slider 166 can be manipulated to expand or constrict the loop portion 165. When partially retracted the slider contacts locking cam 167. Locking cam 167 can be manipulated to prevent or allow further constriction of loop portion 165. The manipulation of lock mechanism 168 can orient the locking cam 167 in the “locked” or “unlocked” position. When unlocked, slider 166 can pass locking cam 167 and engage with arm 169 of cutting handle 170. The application of force depressing cutting handle 161 engages arm 169 with slider 166 and fully retracts the slider towards the rear of the handle portion 161. When cutting handle 170 is fully depressed the loop 165 is fully retracted into tip portion 164, thereby severing any appropriate component disposed in the loop 165.

Referring to FIG. 8C, an embodiment of handle portion 171 can include a slider 166 coupled to loop portion 165. The slider 166 can be manipulated to expand or constrict the loop portion 165. When cutting handle 170 is fully depressed the loop 165 is fully retracted into tip portion 164, thereby severing any appropriate component disposed in the loop 165.

In another embodiment, tip cutter 160 can include an outer sleeve 163 that can be longitudinally translatable along catheter body 162. Extending or retracting outer sleeve 163 while maintaining loop portion 165 in place has the effect of “closing” loop portion 165, and can be used as a quick check prior to actually closing loop portion 165 or to easily adjust the amount loop 165 is open during tracking of tip cutter 160 to a desired location.

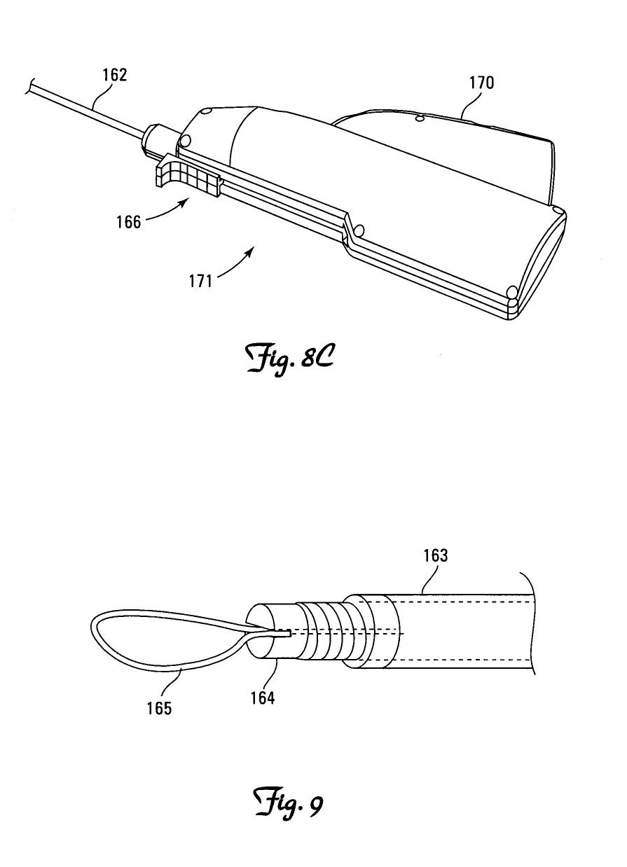

FIG. 9 depicts an embodiment of a tip cutter with a support coil extending inside the length of outer sleeve 163. In one embodiment wire loop 165 can have an approximate diameter of 0.010 inches.

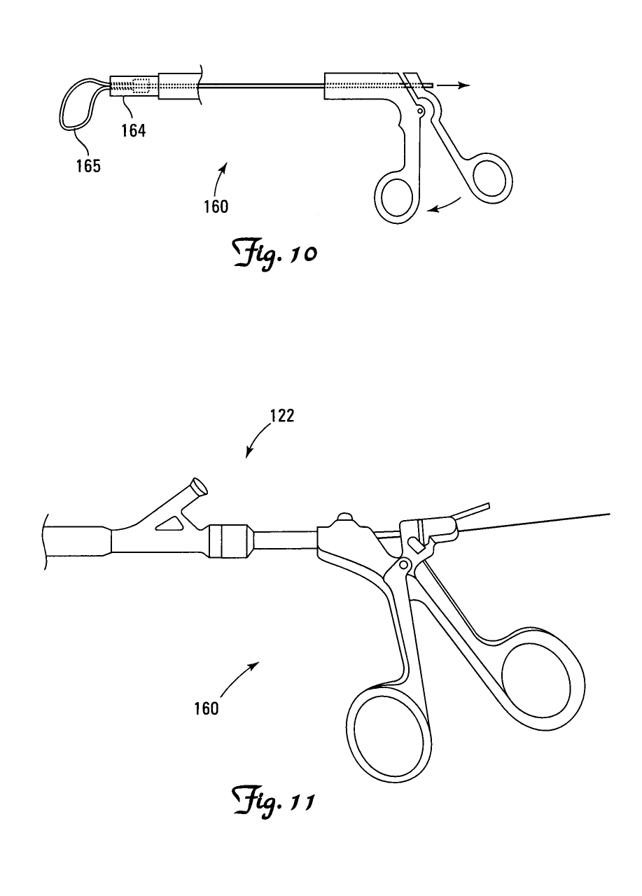

FIGS. 10-11, depict an embodiment of a tip cutter 160 with a generally scissors-handle style actuation mechanism. The actuation of the scissors-handle towards each other retracts an internal connecting wire that retracts loop 165 into tip 164. This handle can be substituted for the handle portion 161 of FIGS. 8A-8B.

The following describes an embodiment of a method of extracting an IID 100 implanted within a patient using the tools described herein. The IID 100 is anchored superior to the heart, such as in the subclavian vein, and extends down into the superior vena cava and extends at least partly into the inferior vena cava. The IID 100 includes a lead 104 integrated with the inferior end of the elongated device body, the lead being looped from the inferior end and extending up into the right atrium and down into the right ventricle. Preferably the removal takes place with the benefit of fluoroscopy visualization or a similar process for real-time internal visualization of the patient.

While the embodiments are described with respect to specific steps and tools, it will be understood that other embodiments of the method of extracting can be used in various combination of some or all of the steps, and with various tools or alternatives to those tools. Although the embodiment of the method of extracting is described with respect to a method performed by a surgeon or medical professional, or by providing instructions and tools to be used by a surgeon or medical professional, it will also be understood that various of the steps or tools may be used either in conjunction with, or directly by, a robotic surgical system, such as the da Vinci robotic surgical system.

First, femoral access is created and an introducer sheath is inserted to maintain vessel access (such as the introducer sheath described and depicted in U.S. Application No. 2009/0192579). Next, safety sheath 120 is delivered through the introducer sheath and advanced into the inferior vena cava toward the inferior end of the IID. Safety sheath 120 is positioned proximate, but spaced a short distance from, the looped section of the IID lead 104.

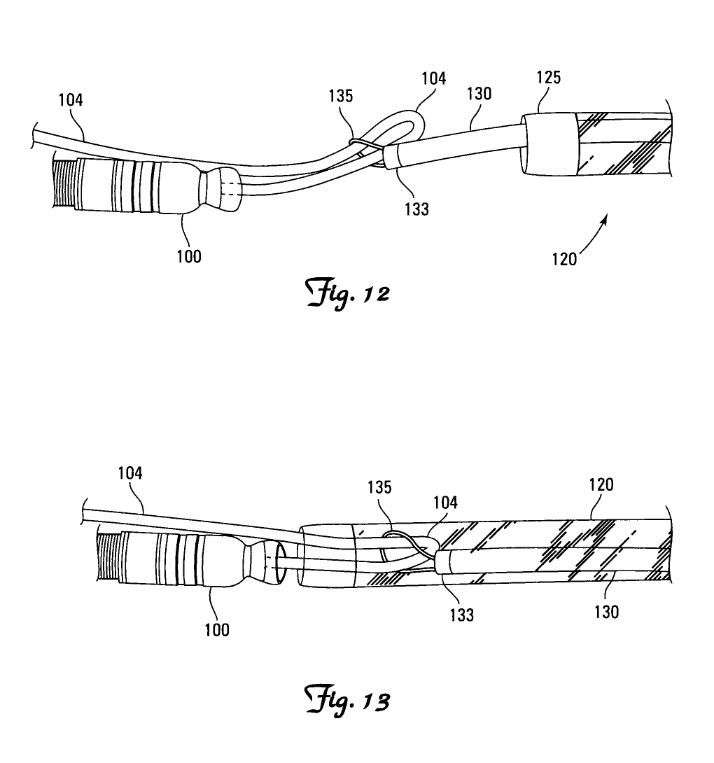

Lead snare 130 is delivered into safety sheath 120 and positioned proximate the captured portion of the looped portion of the lead 104. Using handle portion 131, loop portion 135 is opened large enough to snare the lead 104, as in FIG. 12. Loop portion 135 is then closed against tip portion 134 to grasp the lead 104.

Safety sheath 120 is then advanced further to cover the snared lead such that the lead 104 and snare 130 are within tubular shaft 124, as depicted in FIG. 13. Loop portion 135 of snare 130 can then be opened and withdrawn from the patient, leaving the lead 104 looped in tubular shaft 124. Alternatively the snare 130 can remain in position, thereby holding the lead 104 within the interior of the safety sheath 120.

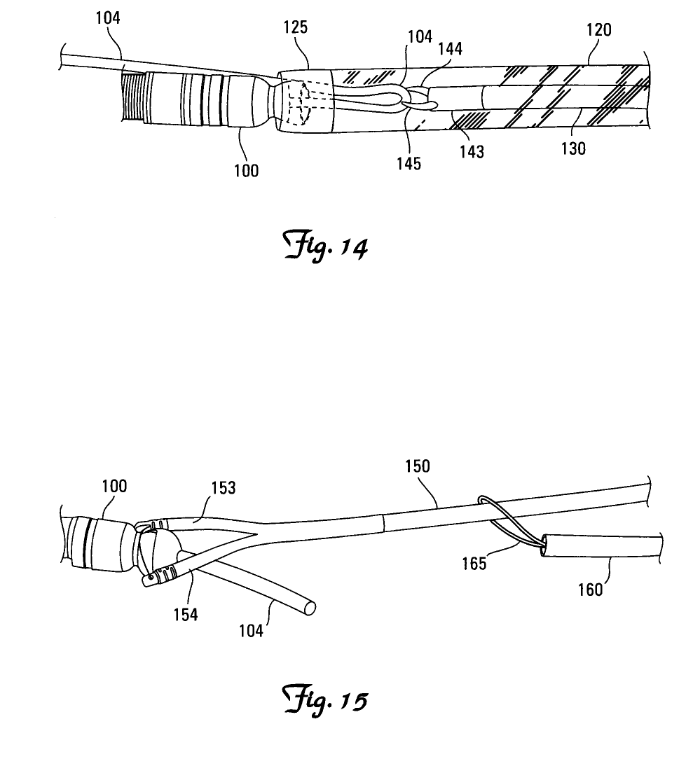

Next, lead cutter 140 is placed into safety sheath 120 and advanced proximate the lead 104 as depicted in FIG. 14. Blades 134 and 135 are opened, lead cutter 140 is advanced against the lead 104, and blades 134, 135 are closed to sever the lead. The cutting of the lead 104 takes place entirely within the safety sheath 120. Lead cutter 140 is then withdrawn from the patient. Safety sheath 120 can also then be withdrawn from the patient.

Tip cutter 160 is pre-loaded over the device snare 150, as depicted in FIG. 15. Tip cutter 160 and device snare 150 are placed into the introducer sheath and advanced toward the proximal end of the IID 100. Handle portion 151 is operated to open loop portion 155, and snare 150 is advanced onto the proximal end of the IID 100. Loop portion 155 is closed securely onto the IID 100, as depicted in FIG. 15. Snare 150 can grasp the body portion of the IID 100, the lead transition portion, or the severed lead stub. At this point, snare 150 can optionally be used to push, pull, and/or rotate IID to attempt to free any thrombus that has formed on the IID.

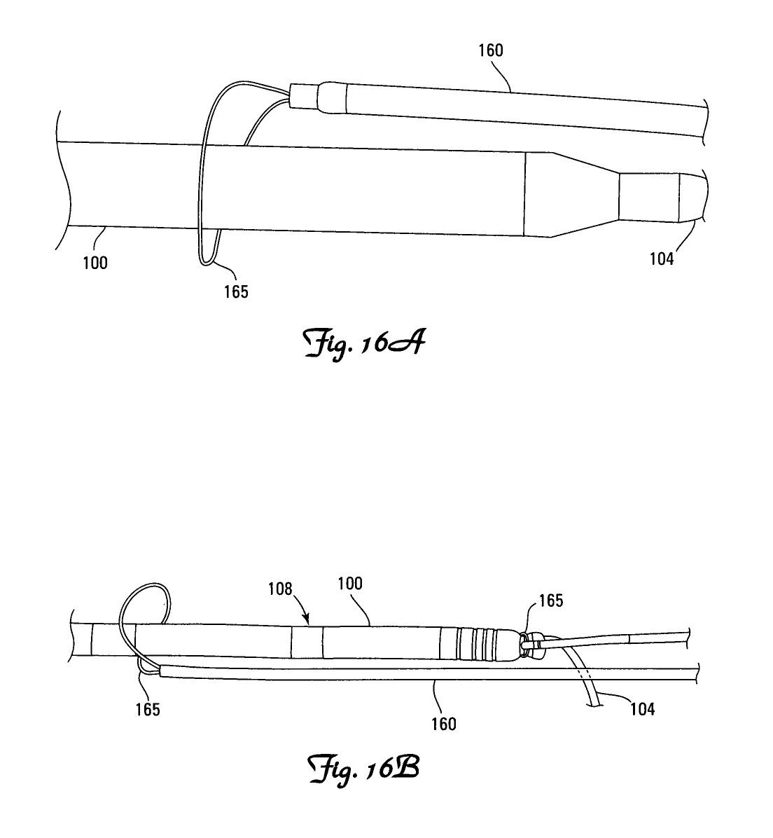

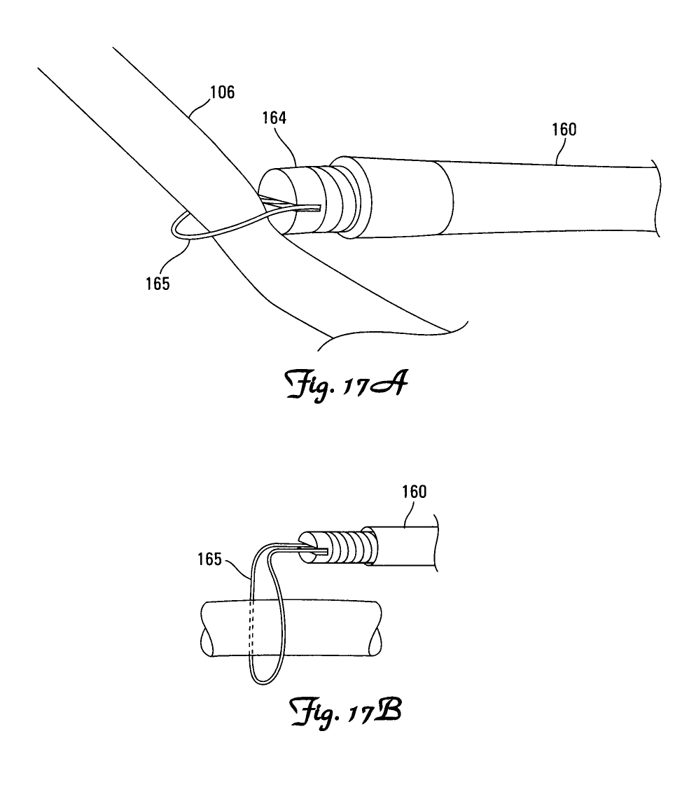

Tip cutter 160 is advanced over snare 150, then over the IID 100 as depicted in FIGS. 16A and 16B, until reaching the distal tip 106 of the IID 100 proximate the anchor. The size of loop portion 165 can be adjusted if necessary during tracking of tip cutter 160.

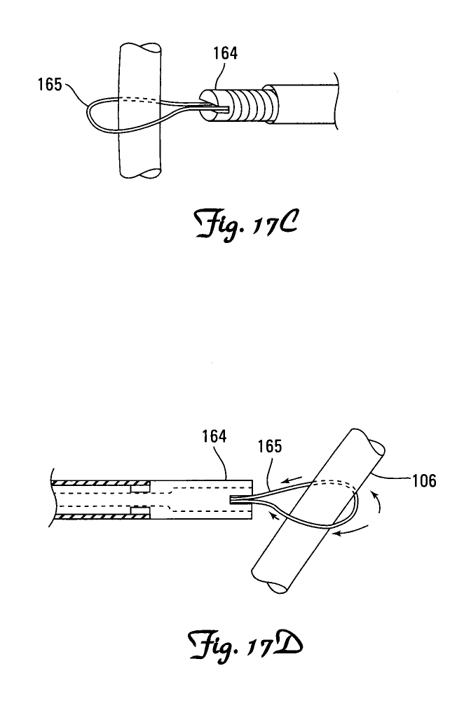

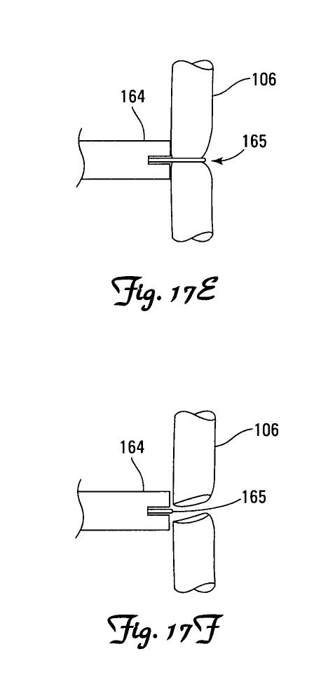

Once loop portion 165 of tip cutter 160 is at a desired location, handle portion 161 is operated to close loop portion 165 as depicted in FIG. 17A, and sever the distal tip 106 of the IID 100, separating the IID device body 102 from its anchor. FIG. 17B depicts tip cutter 160 being deployed along a parallel axis to a lead 104 or IID 100 with loop portion 165 including a right-angle bend to allow the loop portion 165 to circle the lead 104 or IID 100. FIGS. 17C and 17D depict a loop 165 being pulled taught, bringing the lead in contact with tip 164. FIG. 17E depicts loop 165 being fully taught and compressing the lead 104 against the tip 164 at the beginning of a cut. FIG. 17F depicts the loop 165 fully severing the IID tip 106 and being retracted into tip 164 of tip cutter 160. Tip cutter 160 can then be withdrawn from the patient. Finally, device snare 150 is removed from the patient, bringing along the IID 100. The introducer sheath can be removed and the incision closed.

In one embodiment, one or more tools can be introduced into the vasculature to access the severed free end of the lead. For example, an over-the-lead tip cutter 160 can be introduced and advanced to the lead tip. A tip or lead cutter can be introduced into the over-the-lead catheter and the lead body can be cut from the tip. In one embodiment the tip can be left in place after the lead body is removed. Alternatively, any thrombus that has formed on the lead tip can be cut away and the entire lead tip removed. In another embodiment, the severed portion of the cardiac lead is left within the patient and an intravascular stent (such as an AAA stent) is delivered and deployed within the inferior vena cava to trap the severed lead body against the wall of the inferior vena cava.

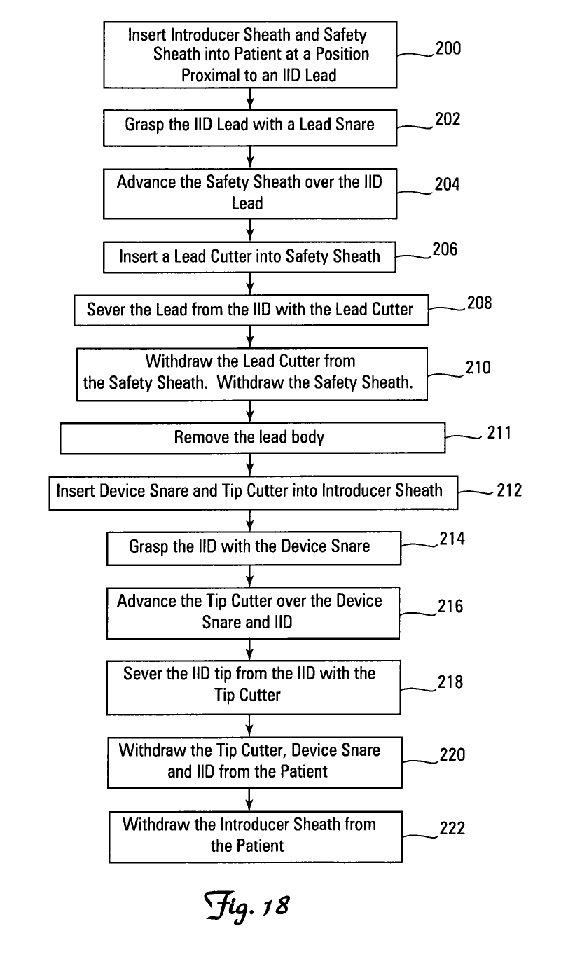

In one embodiment, depicted in FIG. 18, an IID is removed by performing the following steps: 200 Inserting an introducer sheath and safety sheath into a patient at a position proximal to an IID Lead. 202 Grasping the IID lead with a lead snare. 204 Advancing the Safety Sheath over the IID Lead. 206 Inserting a lead cutter into safety sheath. 208 Severing the lead from the IID with the lead cutter. 210 Withdrawing the lead cutter from the safety sheath and withdrawing the safety sheath. 211 Remove the lead body. 212 Inserting device snare and tip cutter into introducer sheath. 214 Grasping the IID with the device snare. 216 Advancing the tip cutter over the device snare and IID. 218 Severing the IID tip from the IID with the tip cutter. 220 Withdrawing the device snare and IID from the patient. 222 Withdraw the introducer sheath from the patient.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention can be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed:

1. A method of extracting an intravascular device anchored in a vasculature of a patient, the device including a device body and a lead coupled to the device body, the lead having a distal end including an electrode, the distal end being fixed in a heart of the patient, the method comprising:
forming an incision in the vasculature;
disconnecting the lead from the device body;
removing the distal end of the lead from the heart;
withdrawing the lead from the patient;
disconnecting the device body from an anchor; and
withdrawing the device body from the patient via the incision.

2. The method of claim 1, wherein disconnecting the lead from the device body comprises cutting the lead.

3. The method of claim 1, wherein removing the distal end of the lead from the heart comprises cutting the distal end of the lead.

4. The method of claim 1, wherein disconnecting the device body from the anchor comprises cutting the device body.

5. The method of claim 1, wherein forming an incision in the vasculature comprises forming an incision in the vasculature at a location inferior of the heart of the patient.

6. The method of claim 1, further comprising:
introducing a safety sheath into the vasculature;
advancing the safety sheath proximate the lead;
manipulating the safety sheath and/or the lead such that a portion of the lead is within the safety sheath; and
disconnecting the lead from the device body by cutting the lead body from within the safety sheath.

7. The method of claim 6, wherein manipulating the safety sheath and/or the lead comprises grasping the lead with a snare and withdrawing a portion of the lead into the safety sheath.

8. The method of claim 1, wherein disconnecting the device body from an anchor comprises:
advancing a cutting tool having a closeable loop portion over the device body to a desired location;
causing the loop portion to close around the device body at the desired location, thereby cutting the device body and separating the device from the anchor.

9. A system for extracting an intravascular device anchored in a vasculature of a patient, the device including a device body and a lead coupled to the device body, the lead having a distal end including an electrode, the distal end being fixed in a heart of the patient, the system comprising:
a safety sheath configured to be temporarily introduced into the vasculature of the patient;
a lead snare having a closeable snare portion, the lead snare configured to releasably capture a portion of the lead;
a lead cutter having a cutting mechanism, the lead cutter configured to sever the lead;
a device snare having a closeable snare portion, the device snare configured to releasably grasp the device body of the intravascular device; and
a tip cutter having a closeable loop portion, the loop portion configured to be advanceable over the device body.

10. A method of extracting an intravascular device anchored in a vasculature of a patient, the device including a device body and a lead coupled to the device body, the lead having a distal end including an electrode, the distal end being fixed in a heart of the patient, the method comprising:
providing a safety sheath, a snare, a lead cutter and a tip cutter; and
providing instructions for extracting the intravascular device, including:
forming an incision in the vasculature;
positioning the safety sheath within the vasculature and proximate the lead;
grasping the lead with the snare and pulling a portion of the lead within the safety sheath;
cutting the lead with the lead cutter;
grasping the device body with the snare;
advancing the tip cutter over the device body to a desired location; and
cutting the device body to disconnect the device body from an anchor.

11. The method of claim 10, wherein providing instructions further comprises:
advancing the tip cutter over the lead to the distal end of the lead;
actuating the tip cutter to cut the distal end of the lead from the heart; and
removing the lead from the patient.

12. A method of extracting an intravascular device anchored in a vasculature of a patient, the device including a device body and a lead coupled to the device body, the lead having a distal end including an electrode, the distal end being fixed in a heart of the patient, the method comprising:
obtaining access to the vasculature;
disconnecting the lead from the device body;
disconnecting the device body from an anchor; and
withdrawing the device body from the patient.

13. The method of claim 12, further comprising:
removing the distal end of the lead from the heart; and
withdrawing the lead from the patient.

14. The method of claim 12, further comprising:
deploying a stent within the vasculature to secure the lead against a vessel wall.
